(19) United States
(12) Patent Application Publication
(10) Pub. No.: US 2020/0246170 A1
(43) Pub. Date: Aug. 6, 2020

(54) Orthopedic Support Device

(71) Applicant: East Coast Orthotic & Prosthetic Corp., Deer Park, NY (US)

(72) Inventors: Vincent Benenati, Dix Hills, NY (US); Johnson Chen, Deer Park, NY (US); Gregory Ahmad, Deer Park, NY (US); Hans Telles, Weinstadt-Benzach (DE); Kevin M. Curran, Little Falls, NJ (US)

(73) Assignee: East Coast Orthotic & Prosthetic Corp., Deer Park, NY (US)

(21) Appl. No.: 16/783,706

(22) Filed: Feb. 6, 2020

Related U.S. Application Data
(60) Provisional application No. 62/802,203, filed on Feb. 6, 2019.

(51) Int. Cl. A61F 5/01; A61F 2/70; A61H 9/00

(52) U.S. Cl. CPC A61F 5/0111; A61H 2205/12; A61H 9/0078; A61F 2/70

(57) ABSTRACT

Methods and apparatus are disclosed regarding orthopedic support devices adapted for stabilizing a part of a body of a wearer, in which an integrated pressure modulation mechanism is adapted to be coupled to at least one evacuable bladder containing molded beads. An exemplary orthopedic support device comprises an orthopedic walker adapted for stabilizing a foot of a wearer. The orthopedic walker comprises an integrated adjustable stabilization system comprising an integrated pressure modulation mechanism, a bladder, a plurality of molded beads inside the bladder, and a bladder coupling. The bladder coupling couples the bladder with the integrated pressure modulation mechanism. The integrated pressure modulation mechanism is adapted to evacuate a fluid from the bladder to decrease its internal pressure to a decreased bladder internal pressure, consolidate the plurality of molded beads from a loose arrangement to a rigid arrangement, and maintain the bladder internal pressure at the decreased bladder internal pressure.

### CROSS REFERENCE TO RELATED APPLICATION

This application is related to and claims the benefit of U.S. Provisional Patent Application Ser. No. 62/802,203 (“the '203 application”), titled “Orthopedic Support Device,” which is incorporated by reference herein in its entirety for all purposes.

### BACKGROUND OF THE INVENTION

### Field of the Invention

The invention relates to methods and apparatus of orthopedic stabilization and support, and in a particular embodiment, to an Orthopedic Walker, also referred to as a boot. Proposed uses include orthopedic stabilization applications of limbs and joints, such as for supporting an ankle or lower leg, such as after breaking the ankle or surgery on the lower leg. In other embodiments of the invention, the orthopedic support device may comprise a shoe, such as a running shoe or an orthotic shoe, or a boot, such a hiking boot, a work boot, a ski boot, or a snowboard boot.

### Description of Related Art

The related art includes, for instance, versions of orthopedic stabilization and support devices such as arm braces, wrist braces, ankle braces, leg braces, and knee braces.

The prior art methods and devices include the use of rigid structures limiting the range of motion of a body part, with non-rigid padding or cushioning placed between the rigid structure and the body part, to conform to the body part and improve wearer comfort.

In some prior art methods and devices, the padding or cushioning includes bladders that a wearer may inflate, such as when donning the device, to cause the padding or cushioning to enlarge and fill the space between the body part and the rigid structure, wherein air in the bladder flows as a fluid in the bladder to adjust to pressure applied to the bladder as the body part and rigid structure are subjected to pressure. The wearer may then deflate the bladder, such as when removing the device, to shrink the bladder and reduce pressure between the body part and the rigid structure. For instance, U.S. Pat. No. 8,002,724 B2 titled “Circumferential Walker” uses bladders, inflation pumps and valves to regulate bladder inflation and resulting pressure.

In some prior art methods and devices, a bladder may not be inflated or deflated by the wearer, but instead is filled with a fixed volume of fluid, such as air, such as in U.S. Pat. No. 7,401,420 titled “Article of footwear having a fluid-filled bladder with a reinforcing structure.”

In another prior art publication, U.S. Pat. No. 5,577,998 titled “Walking Brace” discloses a bladder filled with resilient foam, and when the bladder valve is open, air is forced to flow out of the bladder if the foam is compressed, such as when a user dons that walking brace, whereas air is sucked into the bladder if the compressed foam is permitted to expand, such as when that user removes the walking brace, such that the compressed foam may be maintained in a compressed state by closing the valve to prevent air from returning into the bladder and returning the foam to a non-compressed state.

In still other prior art methods and devices, a bladder may be filled with molded bodies, beads, or microbeads. For instance, U.S. Patent Publication 2006/0229541 A1 titled “Orthopedic inlay” discloses using an external, non-integrated, removable/detachable pump to evacuate a bladder to induce a partial vacuum in the bladder, wherein the vacuum is maintained by a valve mechanism, and evacuating the bladder causes molded beads in the bladder to retain a shape formed when the molded beads consolidate as the air is removed.

As described below, embodiments of the present invention include the use of a bladder filled with molded beads, in which a partial vacuum may be created and maintained in the bladder to immobilize the molded beads, using methods and structures different from those of the prior art methods and devices.

### BRIEF SUMMARY OF THE INVENTION

The invention is directed to methods and apparatus involving an orthopedic support device having an integrated pressure modulation mechanism that is adapted to be coupled to at least one bladder containing a plurality of molded beads, wherein a partial vacuum in the bladder may be formed, maintained, and removed by respectively decreasing, maintaining, and restoring internal pressure of the bladder using an integrated pressure modulation mechanism. Evacuating a fluid from the bladder decreases a bladder internal pressure to a decreased bladder internal pressure and consolidates the plurality of molded beads from a loose arrangement to a rigid arrangement. Proposed uses include orthopedic stabilization applications of limbs and joints, such as for supporting an ankle or lower leg, such as after breaking the ankle or surgery on the lower leg. In other embodiments of the invention, the orthopedic support device may comprise a shoe, such as a running shoe or an orthotic shoe, or a boot, such a hiking boot, a work boot, a ski boot, or a snowboard boot. The size and scale of the components of the invention will be relative to the embodiments in which they are implemented, such that, for example, a running shoe likely would have a smaller bladder than a hiking boot, a hiking boot likely would have a smaller bladder than a snowboard boot, and a snowboard boot likely would have a smaller bladder than orthopedic walker.

The orthopedic support device may function as a stabilization device adapted for stabilizing a part of a body of a wearer; wherein the stabilization device includes an integrated adjustable stabilization system that comprises the integrated pressure modulation mechanism, the bladder, beads within the bladder, and a valve coupled to the bladder. The bladder may be adapted to be evacuated when the stabilization device is worn on the part of the body; wherein when the bladder is evacuated, the beads adjustably consolidate within the bladder to add rigidity to the bladder, and to form a shape that adjustably conforms to the part of the body. The valve is adapted to be switchable between an open position and a closed position, in which the open position opens the bladder to allow fluid flow into and out of the bladder to modify a bladder internal pressure within the bladder, and the closed position closes the bladder to prevent fluid flow into and out of the bladder and to maintain the bladder internal pressure within the bladder.

In accordance with a first aspect of the invention, an orthopedic support device adapted for stabilizing a part of a body of a wearer is disclosed, wherein the orthopedic support device comprises an integrated adjustable stabilization system, wherein the stabilization system comprises an integrated pressure modulation mechanism that is adapted to be coupled to a bladder, containing a plurality of molded beads inside the bladder, via a bladder coupling. The bladder coupling is adapted to couple the bladder with the integrated pressure modulation mechanism. The integrated pressure modulation mechanism is adapted to evacuate a fluid from the bladder through the bladder coupling to decrease a bladder internal pressure to a decreased bladder internal pressure, consolidate the plurality of molded beads from a loose arrangement to a rigid arrangement, and maintain the bladder internal pressure at the decreased bladder internal pressure.

In accordance with a second aspect of the invention, a method for using an orthopedic support device is disclosed, wherein the orthopedic support device comprises an integrated adjustable stabilization system. The stabilization system comprises an integrated pressure modulation mechanism that is adapted to be coupled to a bladder, containing a plurality of molded beads inside the bladder, via a bladder coupling; wherein the bladder coupling is adapted to couple the bladder with the integrated pressure modulation mechanism. The integrated pressure modulation mechanism is adapted to evacuate a fluid from the bladder through the bladder coupling to decrease a bladder internal pressure to a decreased bladder internal pressure, consolidate the plurality of molded beads from a loose arrangement to a rigid arrangement, and maintain the bladder internal pressure at the decreased bladder internal pressure. The method comprises activating the integrated pressure modulation mechanism to draw fluid into the integrated pressure modulation mechanism. The method may further comprise coupling the integrated pressure modulation mechanism with the bladder. The method additionally may comprise then evacuating the bladder using the integrated pressure modulation mechanism to consolidate the plurality of molded beads from the loose arrangement to the rigid arrangement, and maintaining the bladder in an evacuated state having the decreased bladder internal pressure.

In accordance with a third aspect of the invention, a method is disclosed wherein the aforementioned orthopedic support device is used to stabilize a part of a body of a wearer, and wherein the method comprises donning the orthopedic support device on the part of the body of the wearer, evacuating the bladder, and maintaining the bladder in the evacuated state. The method further may comprise releasing the vacuum from the bladder by changing the bladder from the evacuated state and removing the orthopedic support device from the wearer.

In accordance with a fourth aspect of the invention, an orthopedic support device is disclosed wherein the aforementioned integrated adjustable stabilization system further comprises a reservoir, and the reservoir is adapted receive fluid evacuated from the bladder.

In accordance with a fifth aspect of the invention, an apparatus and a method are disclosed in which the integrated adjustable stabilization system comprises a rapid-transfer system, and the method comprises using the rapid-transfer system to quickly transfer a partial vacuum from an integrated pressure modulation mechanism to a bladder to consolidate molded beads in the bladder. The rapid-transfer system comprises a reservoir, and the method further comprises forming a decreased reservoir internal pressure, before or after donning the orthopedic support device. The method further comprises triggering a release of the decreased reservoir internal pressure from the reservoir to the bladder to quickly evacuate the bladder.

In accordance with a sixth aspect of the invention, an apparatus and a method are disclosed in which the integrated adjustable stabilization system comprises a thermally-regulatable system, and the method comprises using the thermally-regulatable system to thermally regulate fluid, which is transferred between an integrated pressure modulation mechanism and a bladder, and is evacuated from the bladder to consolidate molded beads in the bladder. The thermally-regulatable system also may comprise a reservoir, a thermal control device, and a circulation device, and the method further comprises controlling the fluid temperature and circulating the fluid within the system.

In accordance with a seventh aspect of the invention, an apparatus and a method are disclosed in which the integrated adjustable stabilization system comprises a pressure-cycling system, and the method comprises using the pressure-cycling system to cycle pressure of, and control circulation of, fluid transferred between an integrated pressure modulation mechanism and a bladder, and evacuated from the bladder to consolidate molded beads in the bladder. The pressure-cycling system also comprises a reservoir, a circulation device, and a pressure-cycling device, and the method further comprises cycling the pressure of the fluid, and controlling the circulation of the fluid, within the system.

Further aspects of the invention are set forth herein. The details of exemplary embodiments of the invention are set forth in the accompanying drawings and the description below. Other features, objects, and advantages of the invention will be apparent from the description and drawings, and from the claims.

### BRIEF DESCRIPTION OF THE DRAWINGS

By reference to the appended drawings, which illustrate exemplary embodiments of this invention, the detailed description provided below explains in detail various features, advantages and aspects of this invention. As such, features of this invention can be more clearly understood from the following detailed description considered in conjunction with the following drawings, in which the same reference numerals denote the same, similar or comparable elements throughout. The exemplary embodiments illustrated in the drawings are not necessarily to scale or to shape and are not to be considered limiting of its scope, for the invention may admit to other equally effective embodiments having differing combinations of features.

FIG. 1 shows a block diagram of an exemplary embodiment of the invention depicting an orthopedic support device having an integrated adjustable stabilization system comprising an integrated pressure modulation mechanism coupled to a bladder containing molded beads.

FIG. 2 shows a block diagram of another exemplary embodiment having an openablc and closable system comprising an integrated evacuation pump that is openable to atmospheric air.

FIG. 3 shows a block diagram of another exemplary embodiment having a non-openable closed system comprising an integrated evacuation pump coupled to a reservoir and to a bladder, and the reservoir and the bladder are coupled.

FIG. 4 shows a block diagram of another exemplary embodiment having an integrated evacuation pump coupled to a reservoir.

FIG. 5 shows a block diagram of an exemplary embodiment depicting a non-openable closed system comprising a bladder coupled to an integrated pressure modulation mechanism that comprises a volume modulation mechanism.

FIGS. 6A-6C show block diagrams of exemplary stages of a volume modulation mechanism respectively comprising an initial volume, an increased volume, and a decreased volume, and wherein the volume modulation mechanism includes an optional integrated reset mechanism that is adapted to return to a steady-state shape, which is neither compressed nor expanded, from an expanded shape and from a compressed shape.

FIG. 7 shows a block diagram of another exemplary embodiment having a rapid-transfer system comprising a volume modulation mechanism that includes a trigger valve and a syringe-style piston having a rigid cylinder and a lockable plunger adapted to create a decreased internal pressure within the volume modulation mechanism, whereby the decreased internal pressure may be transferred to a bladder upon triggering the trigger valve and thereby evacuate fluid from the bladder.

FIG. 8 shows a block diagram of another exemplary embodiment having a thermally-regulatable system comprising an integrated pressure modulation mechanism that is coupled to a bladder and that comprises a reservoir, a thermal control device, and a circulation device, which are adapted to control circulation of a thermally-regulated fluid between the bladder and the integrated pressure modulation mechanism.

FIG. 9 shows a block diagram of another exemplary embodiment having a pressure-cycling system comprising an integrated pressure modulation mechanism that is coupled to a bladder and that comprises a reservoir, a circulation device, and a pressure-cycling device, which are adapted to cycle pressure of, and control circulation of, a fluid between the bladder and the integrated pressure modulation mechanism.

FIGS. 10A-10F show block diagrams of exemplary combinations of bladders and molded beads, including interconnected beads, groups of beads, strings of beads, matrices of beads, loose arrangements of beads, rigid arrangements of beads, beads within mesh pockets, and beads within a bladder having a lattice.

FIG. 11 shows a front side perspective view of an exemplary embodiment of the invention comprising an orthopedic walker having an integrated evacuation pump and integrated bladder valve.

FIG. 12 shows a rear side perspective view of the orthopedic walker shown in FIG. 11.

FIG. 13 shows a front elevation view of the orthopedic walker shown in FIG. 11.

FIG. 14 shows a side elevation view of the orthopedic walker shown in FIG. 11.

FIG. 15 shows a front side perspective view of an exemplary orthopedic walker having an exemplary evacuation pump and an exemplary bladder valve integrated into the exemplary orthopedic walker.

FIG. 16 shows a side perspective view of the exemplary evacuation pump with an integrated bladder valve that are integrated into the orthopedic walker depicted in FIG. 15.

FIG. 17 shows a side elevation view of the orthopedic walker depicted in FIG. 15.

### LISTING OF DRAWING REFERENCE NUMERALS

an orthopedic support device 1000
   an orthopedic walker 1100
      a front side 1110 of an orthopedic walker
      a back side 1120 of an orthopedic walker
      a hard shell assembly 1130
      an interconnecting part 1140
      an adjustable strap 1150
an integrated adjustable stabilization system 100
   an openable and closable system 110
   a non-openable closed system 120
   a rapid-transfer system 130
   a thermally-regulatable system 140
   a pressure-cycling system 150
a bladder 200
   a molded bead 210
   a loose arrangement 210′ of a plurality of beads 210
   a rigid arrangement 210″ of a plurality of beads 210
      an interconnected bead 212
      a single 214/a bead single 214
      a double 216/pair/a bead double 216
      a triple 218/a bead triple 218
   a connection 220 (e.g., a string, a thread, a fiber, a filament, a molded interconnection) between beads
   a string 230 of interconnected beads
   a matrix 240 of interconnected beads
   a lattice 250
   a continuous weld 260
   a periodic weld 270 (e.g., a mid-point weld, a discontinuous weld, an intermittent weld)
   a non-welded gap 280
   a mesh pocket 290
an integrated pressure modulation mechanism 300
   an integrated bladder valve 310 (e.g., a ball valve, a ball-bearing valve, a compression valve, a pinch valve, a disc valve, a spring valve, a flap valve, a screw valve, a rotation valve, a push valve, a pull valve)
      an air intake 312
      a threaded cylinder 314
      a threaded cap 316
   an integrated evacuation pump 320 (e.g., a bulb pump, a piston pump, a syringe-style pump, a ratchet pump, a screw pump)
      a bulb 320′
      an intake port 322
      a pump inflow valve 324
      an output port 326
      a finger grip 326′
      a pump outflow valve 328
      a pump button 328′
a fluid 400
   a gas 410 (e.g., atmospheric air 410)
   a liquid 420 (e.g., water, oil, or hydraulic fluid)
a fluid coupling 500 (e.g., tubes, tubing, channels, pipes)
   a pump-to-bladder coupling 510
   a pump-to-reservoir coupling 520
   a reservoir-to-bladder coupling 530
   a valve-to-coupling connection 540
a reservoir 600
   a reservoir inflow valve 610
   a reservoir outflow valve 620
a volume modulation mechanism 700
   a volume 710 of the closed system
      an initial volume 712
      an increased volume 714
      a decreased volume 716
   a volume modulator 720
   an integrated reset mechanism 730
      a neutral, steady-state shape 732
      an expanded shape 734
      a compressed shape 736
   a syringe-style piston 740
      a rigid cylinder 742
      a lockable plunger 744
      a trigger valve 746 (e.g., a transfer valve or a release valve, with a trigger, button, handle, etc.)
a thermal control device 800 (e.g., a heating device, a cooling device, a heating/cooling device, or a heat exchanger)
a circulation device 900
   an electric coupling 910
   a pressure-cycling device 920

### DETAILED DESCRIPTION OF THE INVENTION

The invention is directed to methods and apparatus involving an orthopedic support device having an integrated adjustable stabilization system including an integrated pressure modulation mechanism that is adapted to be coupled to at least one bladder containing a plurality of molded beads, wherein a partial vacuum in the bladder may be formed, maintained, and removed by respectively decreasing, maintaining, and restoring internal pressure of the bladder using the integrated pressure modulation mechanism. Evacuating a fluid from the bladder decreases a bladder internal pressure to a decreased bladder internal pressure and consolidates the plurality of molded beads from a loose arrangement to a rigid arrangement.

The orthopedic support device may function as a stabilization device adapted for stabilizing a part of a body of a wearer; wherein the stabilization device includes an integrated adjustable stabilization system that preferably includes the integrated pressure modulation mechanism, the bladder, beads within the bladder, and a valve coupled to the bladder. The bladder is adapted to be evacuated when the stabilization device is worn on the part of the body; wherein when the bladder is evacuated, the beads adjustably consolidate within the bladder to add rigidity to the bladder, and to form a shape that adjustably conforms to the part of the body. The valve is adapted to be switchable between an open position and a closed position, in which the open position opens the bladder to allow fluid flow into and out of the bladder to modify a bladder internal pressure within the bladder, and the closed position closes the bladder to prevent fluid flow into and out of the bladder and to maintain the bladder internal pressure within the bladder.

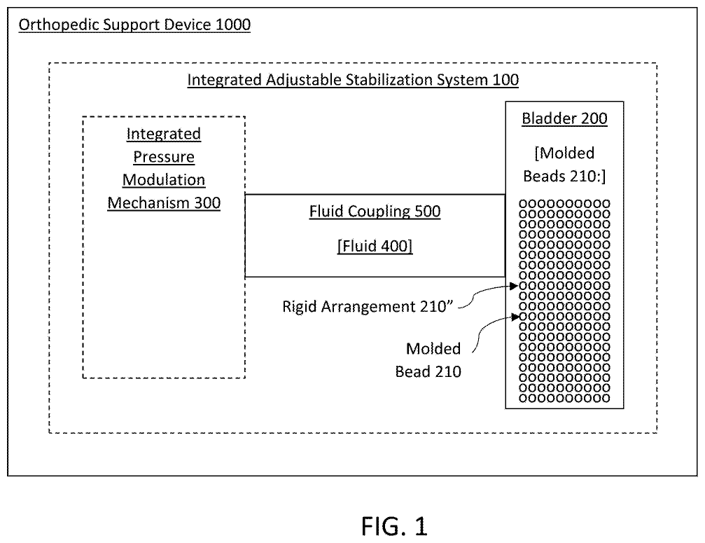

FIG. 1 shows a block diagram of an exemplary embodiment of the invention depicting an orthopedic support device 1000 having an integrated adjustable stabilization system 100 comprising a bladder 200, molded beads 201 within the bladder 200, an integrated pressure modulation mechanism 300, a fluid 400 within the system 100, and a fluid coupling 500 that couples the bladder 200 and the integrated pressure modulation mechanism 300. The bladder 200 and/or fluid coupling 500 may be adapted to be decoupled from and recoupled with the integrated pressure modulation mechanism 300. The bladder 200 and/or fluid coupling 500 also may be adapted to be detached from and reattached to the system 100 and/or device 1000. In FIG. 1, a rigid arrangement 210′ of molded beads is depicted, which arises when the bladder 200 is evacuated of fluid 400 and has a decreased bladder internal pressure.

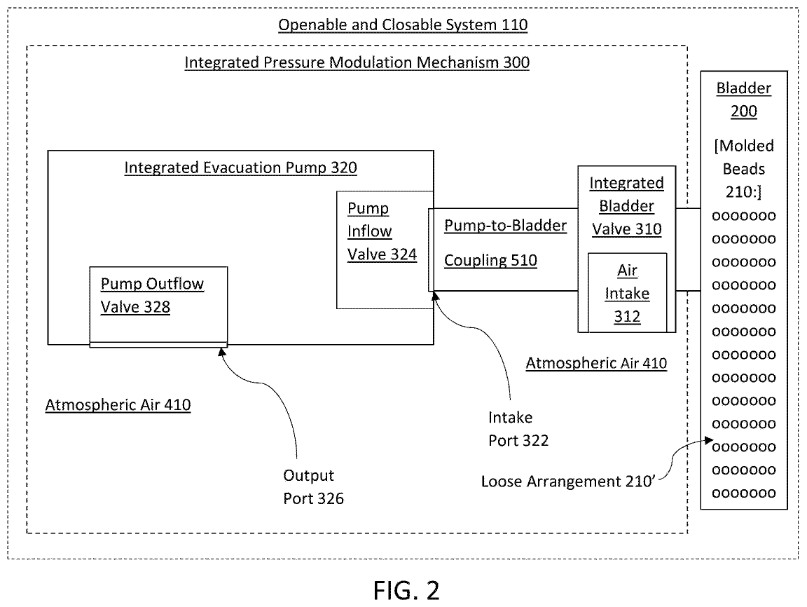

In some embodiments of the present invention, the bladder 200 and the integrated pressure modulation mechanism 300 form an openable and closable system 110. FIG. 2 shows a block diagram of another exemplary embodiment of an orthopedic support device 1000 having an openable and closable system 110 that is openable to atmospheric air 410. The system 110 shown includes an integrated pressure modulation mechanism 300 comprising an integrated bladder valve 310, an air intake 312, and an integrated evacuation pump 320 having an intake port 322, a pump inflow valve 324, and output port 326, and a pump outflow valve 328. The system 110 shown also includes a pump-to-bladder coupling 510 that couples the bladder 200 with the integrated evacuation pump 320, and the coupling 510 may be opened or closed using the integrated bladder valve 310. In FIG. 2, a loose arrangement 210″ of molded beads is depicted, which arises when the bladder 200 is not evacuated of fluid 400 and has a non-pressurized bladder internal pressure (e.g., the bladder internal pressure approximates atmospheric pressure) or an increased bladder internal pressure caused be partial inflation with fluid 400.

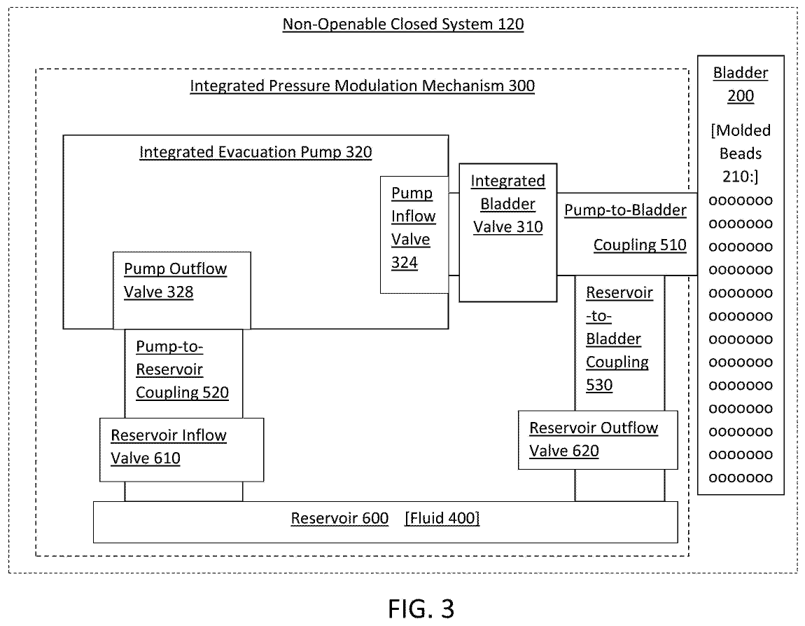

In other embodiments of the present invention, the bladder 200 and the integrated pressure modulation mechanism 300 form a non-openable closed system 120. FIG. 3 shows a block diagram of another exemplary embodiment having a non-openable closed system 120 comprising an integrated evacuation pump 320 coupled to a bladder 200 by a pump-to-bladder coupling 510, and to a reservoir 600 by a pump-to-reservoir coupling 520. The reservoir 600 and the bladder 200 are coupled by a reservoir-to-bladder coupling 530. The reservoir 600 is depicted as having a reservoir inflow valve 610 and a reservoir outflow valve 620 that respectively regulate flow of fluid 400 into and out of the reservoir 600.

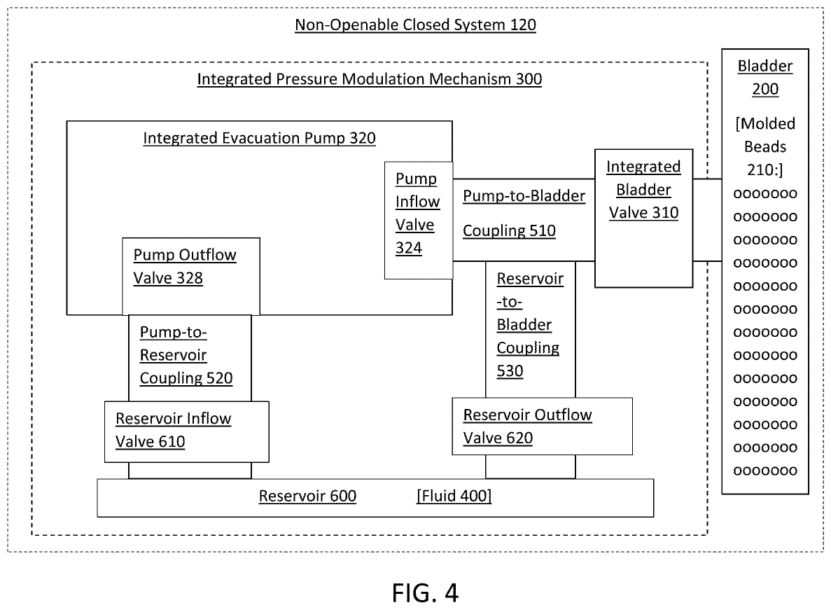

FIG. 4 shows a block diagram of another exemplary embodiment of an orthopedic support device 1000 having non-openable closed system 120 similar to that of FIG. 3, in which an integrated evacuation pump 320 is coupled to a reservoir 600.

In FIG. 3, the reservoir-to-bladder coupling 530 is coupled to the pump-to-bladder coupling 510 between the bladder 200 and integrated bladder valve 310. In FIG. 4, by contrast, the reservoir-to-bladder coupling 530 is coupled to the pump-to-bladder coupling 510 between the integrated bladder valve 310 and the integrated evacuation pump 320. Each of the configurations of FIG. 3 and FIG. 4 has its own advantages and disadvantages, and the nature of a particular orthopedic support device 1000 will help determine which configuration is more effective, more efficient, less expensive, etc., for that particular orthopedic support device 1000.

The integrated pressure modulation mechanism 300 may comprise one of several variations. Pressure within the bladder 200 may be formed by a fluid 400, such as a gas 410 or a liquid 420. Pressure formed by a gas 410 may comprise, for example, atmospheric air 410 in an openable and closable system 110 that is openable and closable to atmospheric air 410. Pressure formed by a liquid 420 may comprise, for example, water, oil, or hydraulic fluid, preferably within a non-openable closed system 120. Pressure may be formed by a liquid 420 within an openable and closable system 110, such as water in and/or added to the system 110, in which case extra care is advisable to reduce risks of the liquid 420 spilling during the decreasing and restoring of internal pressure, thereby making a mess for the wearer.

In some embodiments of the present invention, such as depicted in FIG. 2, the integrated pressure modulation mechanism 300 comprises an integrated evacuation pump 320 and an integrated bladder valve 310. In some embodiments, the integrated bladder valve 310 may be opened and closed, and when the integrated bladder valve 310 is opened to a opened position to form a opened pump-to-bladder coupling 510 between the bladder 200 and the integrated pressure modulation mechanism 300, the integrated pressure modulation mechanism 300 may be used to decrease the internal pressure of the bladder 200 and within the system 100, thereby immobilizing the molded beads 210 contained within the bladder 200, and then the integrated bladder valve 310 may be closed to a closed position to form a closed coupling 510 between the bladder 200 and the integrated pressure modulation mechanism 300 and maintain the decreased internal pressure of the bladder 200 and immobilization of the molded beads 210 to maintain a rigid arrangement 210″.

As shown in FIG. 2, the integrated pressure modulation mechanism 300 may comprise an integrated evacuation pump 320 coupled with the bladder 200 at an intake port 322 of the integrated evacuation pump 320 and having an integrated bladder valve 310 between the bladder 200 and the integrated evacuation pump 320, wherein the integrated bladder valve 310 may open and close the coupling 510 of the bladder 200 and the integrated evacuation pump 320. The integrated evacuation pump 320 also comprises a pump inflow valve 324 that regulates inflow, and a pump outflow valve 328 that regulates outflow, of fluid 400 into and out of the integrated evacuation pump 320. A pump activation cycle may comprise activating the integrated evacuation pump 320 (1) to decrease a pump volume from an initial pump volume to a decreased pump volume by opening the pump outflow valve 328 by forcing pump contents out of the integrated evacuation pump 320; and (2) once decreasing the pump volume ceases, the pump outflow valve 328 closes, the integrated pump 320 returns from the decreased pump volume to the initial pump volume by evacuating the bladder 200 by pulling fluid 400 from the bladder 200 through the opened pump inflow valve 324 and into the volume of the integrated pump 320. Repeating this pump activation cycle incrementally decreases the pressure in the bladder 200.

As depicted in FIGS. 3-4, in some embodiments, the integrated pressure modulation mechanism 300 may comprise a reservoir 600, and the fluid 400 of the pump 320 contents forced from the pump volume may be forced into the reservoir 600.

The reservoir 600 may retain fluid 400 evacuated from the bladder 200 when the integrated bladder valve 310 is closed, and the reservoir 600 may release the fluid 400 back to the bladder 200 to increase the pressure in the bladder 200 upon opening the integrated bladder valve 310. The bladder 200 and the integrated pressure modulation mechanism 300 with the reservoir 600 may form an integrated non-openable closed system 120 that contains the fluid 400 that may come from and go back into the bladder 200. The reservoir 600 may be coupled to the integrated evacuation pump 320 using a pump-to-reservoir coupling 520, which may have a reservoir inflow valve 610 to control whether fluid may enter the reservoir 600 from the integrated evacuation pump 320, and allow pressure to equalize between the reservoir 600 and the integrated evacuation pump 320. The reservoir 600 may be coupled to the bladder 200 using a reservoir-to-bladder coupling 530, which may have a reservoir outflow valve 620 to control whether fluid 400 may exit the reservoir 600, enter the pump-to-bladder coupling 510, and possibly allow pressure to equalize between the reservoir 600 and the bladder 200.

As depicted in FIG. 3, the reservoir-to-bladder coupling 530 may couple to the pump-to-bladder coupling 510 without an integrated bladder valve 310 between the reservoir-to-bladder coupling 530 and the bladder 200, which allows fluid 400 and pressure to equalize between the bladder 200 and the reservoir-to-bladder coupling 530, and, if the reservoir outflow valve 620 is open, with the reservoir 600 as well. Alternatively, as shown in FIG. 4, the reservoir-to-bladder coupling 530 may couple to the pump-to-bladder coupling 510 with an integrated bladder valve 310 between the reservoir-to-bladder coupling 530 and the bladder 200, which requires that the integrated bladder valve 310 be open for fluid 400 and pressure to equalize between the bladder 200 and the reservoir 600.

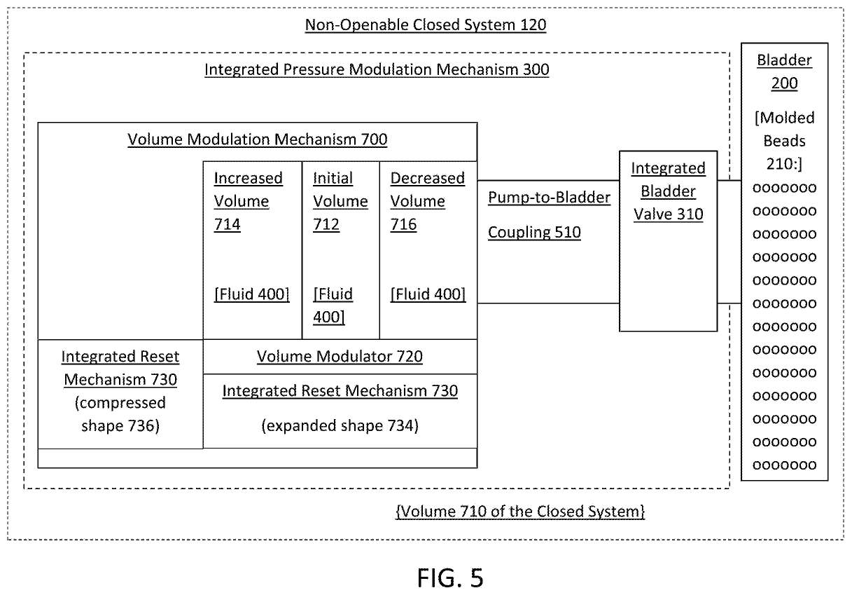

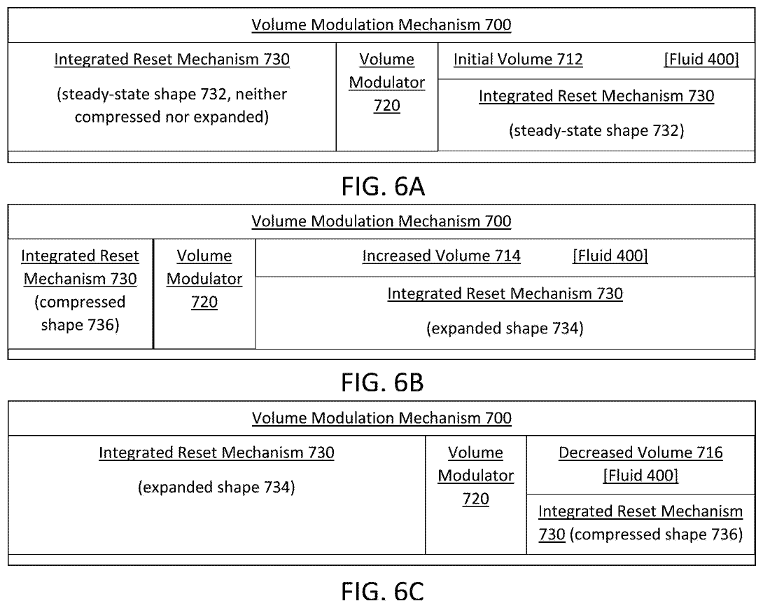

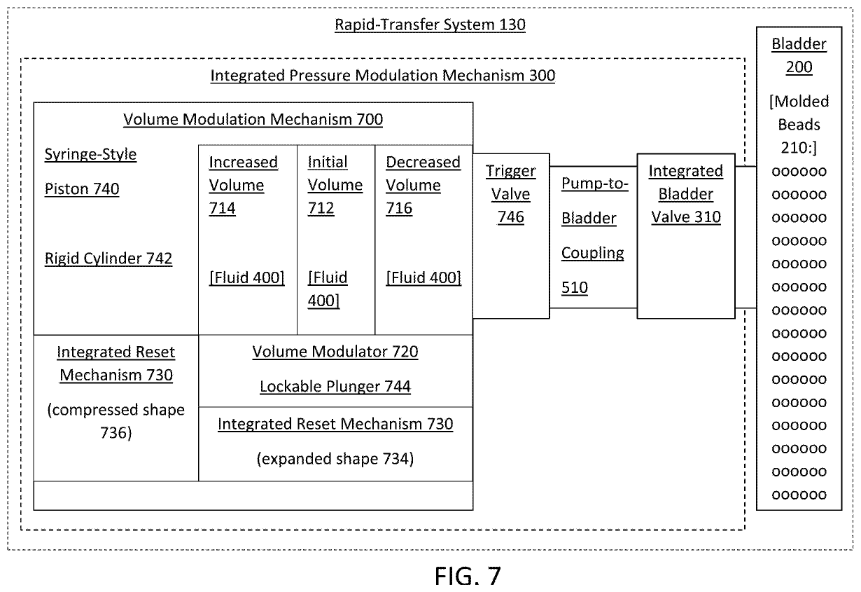

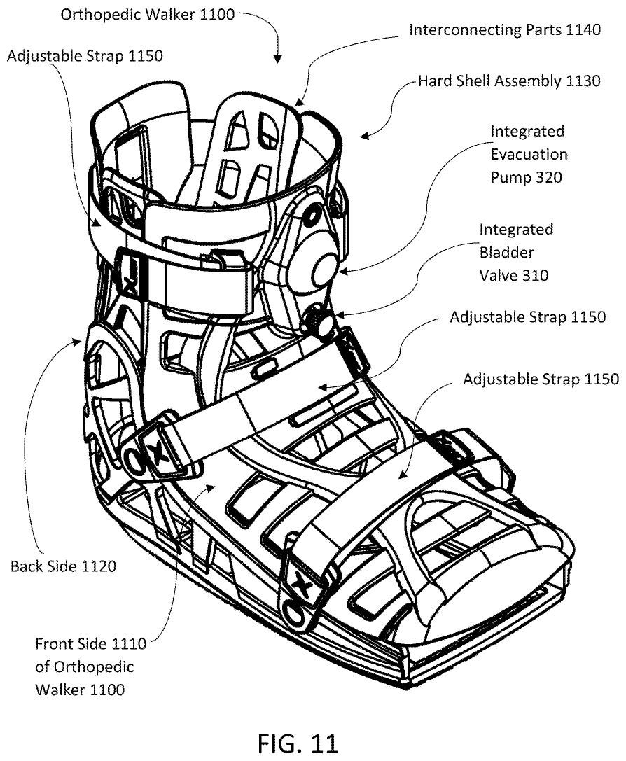

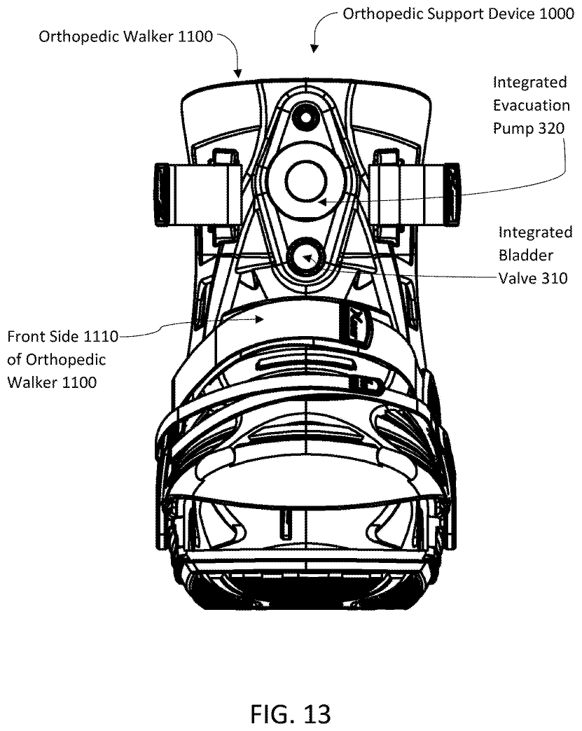

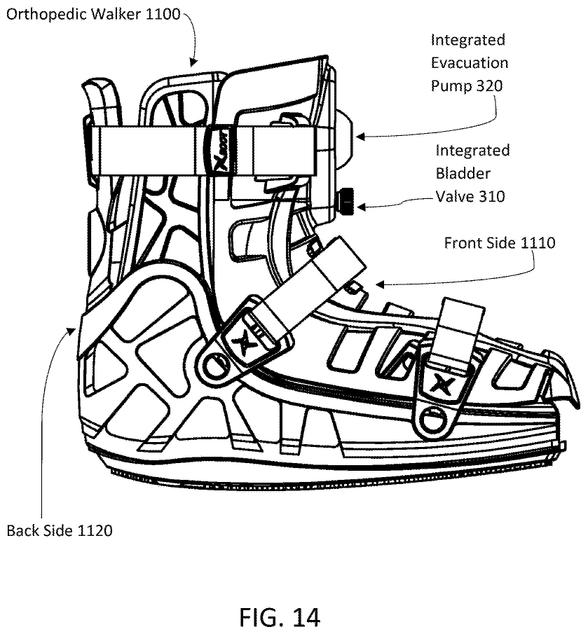

In some embodiments, the evacuation pump 320 may comprise one of a bulb pump, as shown in FIGS. 11-17 (e.g., squeeze the bulb into a compressed shape to force fluid out of bulb, release bulb to draw fluid into the bulb by the force of the bulb returning to its non-compressed shape), a piston pump, as conceptually depicted in FIG. 7 (e.g., a syringe-style pump, in which a user pulls the piston to draw fluid by increasing chamber volume, and pushes the piston to expel fluid by decreasing chamber volume), a ratchet pump, as conceptually depicted in FIGS. 5-6C (e.g., a rotating gear that may increase/decrease a chamber volume to decrease/increase internal pressure within the chamber; in another version: the rotating gear might input, isolate, and output fluid to decrease/increase internal pressure within the bladder), and a screw pump, also as conceptually depicted in FIGS. 5-6C (e.g., similar to a piston pump, using a screw-action piston within a chamber instead of a pull/push-action piston, with a screw-action piston likely creating an integrated locking mechanism to maintain the position and pressure of the screw piston).

FIG. 5 shows a block diagram of an exemplary embodiment of an orthopedic support device 1000 depicting a non-openable closed system 120 comprising a bladder 200 coupled to an integrated pressure modulation mechanism 300 that comprises a volume modulation mechanism 700. In such embodiments of the present invention, the integrated pressure modulation mechanism 300 comprises a volume modulation mechanism 700 adapted to be coupled with and possibly decoupled/detached from the bladder 200. The volume modulation mechanism 700 and the bladder 200 may comprise a non-openable closed system 120 that defines a volume 710 of the closed system 120 and contains a fixed quantity of fluid 400, such that increasing the volume 710 using a volume modulator 720 decreases the internal pressure of the bladder 200, and decreasing the volume 710 using the volume modulator 720 increases the internal pressure of the bladder 200. The integrated pressure modulation mechanism 300 may comprise an integrated bladder valve 310 between the bladder 200 and volume modulation mechanism 700, and closing the integrated bladder valve 310 to a closed position may further stabilize and maintain the decreased internal pressure of the bladder 200.

FIGS. 6A-6C show block diagrams of exemplary stages of a volume modulation mechanism 700 respectively comprising an initial volume 712, an increased volume 714, and a decreased volume 716, and wherein the volume modulation mechanism 700 includes an optional integrated reset mechanism 730 that is adapted to return to a steady-state shape 732, which is neither compressed nor expanded, from an expanded shape 734 and/or from a compressed shape 736.

As conceptually depicted in FIGS. 5-7, the volume modulator 720 may comprise, for example, a piston 740 comprising a cylinder 742 and a plunger 744 in the cylinder 742. The piston 740 may be adapted to have the plunger 744 be positioned at a neutral, steady-state position at which the piston has an initial volume 712. The piston 740 and/or plunger 744 may be pulled or retracted to expand or increase the volume to an increased volume 714 and decrease the internal pressure, wherein the plunger 744 may be secured to an expanded position to maintain the increased volume 714, such as by unscrewing the plunger 744 within the piston 740 to the expanded position, or pulling open the piston 740 and locking the plunger 744 and piston 740 in the expanded position. The piston 740 and/or plunger 744 may be pushed or collapsed to reduce the volume back to the initial volume 712, or beyond to a decreased volume 716 and increase the internal pressure. The piston 740 and/or plunger 744 may be locked in a collapsed position to partially inflate the bladder 200, such as to allow the molded beads 210 to be jostled and repositioned.

In some embodiments, the piston may comprise a regular pole-in-cylinder piston. In other embodiments, the piston may comprise a deformable elastomeric molding that may be expanded or compressed to change the volume defined by the deformable elastomeric molding. The deformable elastomeric molding may be designed to return to and reform its neutral shape when force is not being applied to deform the molding to an expanded or compressed shape.

In some embodiments, a piston may be positioned vertically along a front side 1110 of an orthopedic walker 1100 (e.g., in front a wearer's shin) or along a back side 1120 of an orthopedic walker 1100 (e.g., behind a wearer's calf). For instance, in a system using a vertically positioned piston to evacuate the bladder 200, the piston may function similarly to a syringe that snaps in place in the walker 1100 and that has a downward pointing tip coupled to the bladder 200. A user may rotate and/or snap the syringe-style piston 740 away from the walker 1100, and, with the bladder valve 310 open, adjust the syringe-style piston 740 position to draw fluid 400 out of the bladder 200 to evacuate the bladder 200, or push fluid 400 into the bladder 200 to refill the bladder 200. The user then may close the bladder valve 310 and rotate and/or snap the syringe-style piston 740 back into the walker 1100. Alternatively, the syringe-style piston 740 may be adapted to be used in place on the device 1000 without being removed, rotated or snapped away from or back into the walker 1100.

In some embodiments, the volume modulation mechanism 700 may comprise a plurality of mini pistons arranged on an orthopedic walker 1100, such as above a wearer's instep on a front side 1110, or behind a wearer's calf on a back side 1120, and connected by a cord or cable laces in a fashion similar to shoe laces, such that tightening the laces pulls the mini pistons towards a center line, increases the volume of each mini piston, decreases the internal pressure of the system 120, and evacuates bladder 200, thereby immobilizing the molded beads 210. The laces can be tightened as with regular shoe laces, such as by being manually pulled tight and tied in a bow or knot, or they can be tightened using an integrated winch for a secure adjustment. A winch or ratchet device may be engaged to incrementally tighten the cable or laces, and then disengaged to loosen the cable or laces.

As conceptually depicted in FIGS. 5-7, in some embodiments, the integrated pressure modulation mechanism 300 may comprise an integrated reset mechanism 730 that may return the integrated evacuation pump 320 or volume modulator 720 to an initial volume 712. The integrated reset mechanism 730 may comprise, for example, an elastomeric material that takes a neutral, steady-state shape 732 (e.g., a bulb, or a plane, such as a rubber band, or a foam pad) in the absence of pressure and may shrink or expand in the presence of pressure or force. Another example of an integrated reset mechanism 730 comprises a spring that takes a neutral shape 732 in the absence of pressure, and may be extended to an expanded shape 734, or compressed to a compressed shape 736, in the presence of pressure or force, in one direction or the other.

In some embodiments of the invention, the orthopaedic support device may include one or more types of valves 310, 324, 328, 610, 620, 746, which may comprise, for example, a ball valve, a compression valve, a disc valve, a spring valve, a flap valve, and a screw valve. The choice of valve type depends on the valve's location in, and overall design of and configuration of, a given orthopaedic support device 1000. The disclosures of this present application provide a person of ordinary skill in the art with sufficient information and direction to adequately configure an orthopaedic support device, including the selection and placement of valves, in accordance with the present invention and within the scopes of the attached claims.

A ball valve, also referred to as a ball-bearing valve, may comprise a ball, e.g., a ball bearing of a ball-bearing valve, contained in the valve such that the ball falls, rests, is pushed, or is pulled into a closed position with a lower pressure/vacuum on one side holding the ball in place and a higher pressure on the other side pushing the ball into the closed state. For example, evacuation pumping may exert force to move the ball into an open position by drawing fluid out of the bladder, and stopping the pumping removes the forces holding the ball in the open position, thereby causing the ball to be sucked back into a closed position.

A compression valve, also possibly referred to as a pinch valve, may comprise, for instance, a tube that is compressed or pinched to block the tube, by flattening the tube, and create a closed position of the valve, and when the compression/pinch is removed to unblock the tube, the tube returns to its cylindrical shape, to create an open position of the valve. Alternatively, a compression valve may comprise a tube with a barrier forming a closed position when the barrier is in a neutral shape, and when the tube and barrier are detbrmed from the neutral shape to a deformed shape, a slit in the barrier widens to form an open position to allow fluid to flow through the barrier.

A disc valve, possibly referred to a rotation valve, may comprise a disc, partial sphere, or a rounded bar that may be rotated within a rotation chamber between an open position, opening a passage through the rotation chamber, and a closed position, blocking the passage through the rotation chamber.

A spring valve, also referred to as a push valve or a pull valve (depending on the orientation of the spring), may alternate between open and closed positions as the spring is forced from its neutral shape to a deformed shape. In some embodiments, applying force to the spring opens the valve, while in other embodiments, applying force to the spring closes the valve. Force may be applied to push a button to expand or compress a spring, and/or force may be applied to pull a handle to expand or compress a spring. For example, a spring valve may comprise a spring-actuated component that maintains a resting state of either an open position, in which the spring-actuated component rests in a neutral position or a slightly-expanded-spring position that opens a passage through the valve, and pushing a button to depress the spring-actuated component moves the spring-actuated component into a collapsed-spring position that blocks a passage through the valve. Alternatively, a spring-actuated component may maintain a resting state of a closed position, in which the spring-actuated component rests in neutral position or a slightly-expanded-spring position that closes a passage through the valve, and depressing the spring-actuated component moves the spring-actuated component into a collapsed-spring position that opens a passage through the valve. By placing the spring on the opposite side of the valve, the spring may be forced from a neutral position to an expanded position. Conversely, instead of pushing a button to depress or expand the spring-actuated component, a handle may be pulled to depress or expand the spring-actuated component to open or close the spring valve.

A flap valve may comprise a flap covering a port of the valve, and the flap may be designed to open when pressure is applied in a first direction and to close when pressure is applied in an opposing direction. A flap valve may function similarly to a ball valve in that opening and closing of the valve may be controlled by the application of fluid pressure within a system, and not directly by a button, screw, or handle, for instance. In some embodiments of the present invention, an evacuation pump may comprise a flap valve as a pump inflow valve and as a pump outflow valve, wherein compressing the evacuation pump closes a pump inflow flap on the pump inflow valve while opening a pump outflow flap on the pump outflow valve, forcing fluid out of the pump. Releasing the evacuation pump does the reverse, closing the pump outflow flap on the pump outflow valve while opening the pump inflow flap on the pump inflow valve, drawing fluid into the pump from and out of a bladder to evacuate the bladder.

A screw valve may comprise a threaded cylinder 314 covered by a threaded cap 316, as shown in FIG. 16, wherein the threaded cylinder 314 is coupled to each of two or more sides of a fluid exchange assembly, wherein screwing the threaded cap 316 to the threaded cylinder 314 either closes or opens a coupling between the sides of a fluid exchange assembly. For instance, as shown in FIGS. 11-17, in an openable and closable system 110 that may be opened to exchange atmospheric air, the screw valve 310 may be opened to atmospheric air 410 by unscrewing the threaded cap 316 to an atmosphere-opened position to allow atmospheric air 410 to enter the system 110 and inflate a bladder 200 that has been deflated by an integrated evacuation pump 320, thereby removing the partial vacuum in the bladder 200. Conversely, the screw valve 310 may be closed to atmospheric air 410 by screwing the threaded cap 316 to an atmosphere-closed position to couple the bladder 200 to the pump 320 and to allow air 410 within the bladder 200 to be evacuated by the evacuation pump 320, without the evacuation pump 320 pulling in atmospheric air 410 instead of deflating the bladder 200.

In some embodiments, a screw valve may have more than the two positions mentioned above. For instance, a screw valve might have three positions, wherein (1) unscrewing the threaded cap may form an atmosphere-opened position (opening to atmospheric air the coupling of the fluid exchange between the bladder and the evacuation pump), (2) screwing the threaded cap to a near-fully-screwed position may form an atmosphere-closed position (closing an air intake to atmospheric air of the pump-to-bladder coupling, while keeping open the coupling of the fluid exchange between the bladder and the evacuation pump), and (3) screwing the threaded cap to fully-screwed position that closes the coupling of the fluid exchange between the bladder and the evacuation pump, so as to more securely maintain a reduced internal pressure of an evacuated bladder. Likewise, a bladder valve that closes the coupling between the bladder and the evacuation pump avoids relying on the pump inflow valve not to leak, and thereby avoids incremental pressure increases caused by a possible gradual leak through the pump inflow valve.

In some embodiments, the stabilization system 100 further comprises a release valve, such as for quick release of the vacuum pressure in the bladder 200, and the release valve may comprise an integrated bladder valve 310 or a reservoir outflow valve 620. In an openable and closable system 110, the release valve may allow the fluid 400 (e.g., atmospheric air 410) to flow into the bladder 200 from the adjacent atmosphere, to quickly equalize the bladder internal pressure and an atmospheric pressure adjacent the stabilization device 1000. In a non-openable closed system 120, the release valve may allow the fluid 400 to flow between a reservoir 600 and the bladder 200 to equalize the bladder internal pressure and a reservoir internal pressure as the fluid 400 finds an equilibrium between the reservoir 600 and the bladder 200.

In embodiments in which the bladder 200 and integrated pressure modulation mechanism 300 form an integrated openable and closable system 110 without a reservoir 600, the system 110 comprises a single pressurized chamber (pressurized relative to atmospheric pressure), i.e., the bladder 200, when the bladder 200 is in an evacuated state. During evacuation to reduce the bladder internal pressure, such as pumping action, the pump 320 is temporarily pressurized during the pumping action, but the pump typically ceases to be pressurized once pumping action stops. In an exemplary openable and closable system 110 having a single pressurized chamber, the bladder 200 may exchange air 410 with the atmosphere when the system 110 is open, and only the bladder 200 remains pressurized once the system 110 is closed and pressure modulation ceases (e.g., pumping action is stopped).

In another exemplary openable and closable system 110 having a single pressurized chamber, a pump 320, and a reservoir 600, the bladder 200 may exchange liquid 420 with the reservoir 600, and when the system 110 is open, the reservoir 600 may receive liquid 420 from a liquid source and/or air 410 from the atmosphere. When the system 110 is closed, only the bladder 200 remains pressurized when both the pump-to-bladder coupling 510 is closed and the reservoir-to-bladder coupling 530 is closed from equalizing pressure with the bladder 200. For example, an openable and closable system 110 having a reservoir 600 and a liquid 420 may comprise a pump 320 that cycles the liquid 420 between the bladder 200 and the reservoir 600, and the reservoir 600 may be unpressurized (i.e., at atmospheric pressure) when the bladder 200 is closed, evacuated, or being evacuated.

For instance, the pump 320 may pull liquid 420 from the bladder 200 to pressurize the bladder 200, and push the liquid 420 to the unpressurized reservoir 600. Upon evacuating the liquid 420 from the bladder 200 to pressurize the bladder 200 and form an internal vacuum, the liquid 420 is drawn into the reservoir 600 by the pumping action. A reservoir outflow valve 620 is closed during pumping action to cause the reservoir 600 to retain the liquid 420 drawn from the bladder 200 into the reservoir 600. To reinflate and depressurize the bladder 200, the reservoir 600 may feed (e.g., gravity-fed) the bladder 200 through a reservoir outflow valve 620, in which the reservoir 600 may include the liquid 420, and air 410 above liquid 420, wherein air 410 may equalize pressure with the atmosphere through a gas-permeable, liquid-impermeable barrier.

Once pumping action ceases, only the bladder 200 remains pressurized. When the bladder 200 is not pressurized to form an internal vacuum, and the reservoir outflow valve 620 is open to allow liquid 420 to flow to bladder 200, the liquid 420 forms an equilibrium between the reservoir 600 and the bladder 200.

In embodiments having a reservoir 600, the bladder 200 and the integrated pressure modulation mechanism 300 may comprise a first pressurized chamber, i.e., the bladder 200, and a second pressurized chamber, i.e., the reservoir 600, such as in a non-openable closed system 120. The fluid 400 evacuated from the bladder 200 may be either a gas 410 or a liquid 420. In an exemplary system 120 using a pump 320 to evacuate either a gas 410 or a liquid 420, the system 120 may cycle the fluid 400 from the bladder 200, through the pump 320, into the reservoir 600, and then back into the bladder 200. Because the system 120 is a non-openable closed system 120, the cumulative pressure of the closed system 120 is determined by the quantity of fluid 400 contained in the system 120 relative to the volume 710 defined by the system 120. When the reservoir outflow valve 620 is closed, and fluid 400 is evacuated from the bladder 200, the fluid 400 is pushed into the reservoir 600, thereby decreasing the bladder internal pressure and increasing the reservoir internal pressure. Once the bladder 200 is evacuated, a bladder valve 310 may be closed to secure the decreased bladder internal pressure. Upon opening the reservoir outflow valve 620 and the bladder valve 310, the reservoir internal pressure will force the fluid 400 into the bladder 200 to equalize the pressure within the reservoir 600 and the bladder 200.

FIG. 7 shows a block diagram of another exemplary embodiment of an orthopedic support device 1000 having a rapid-transfer system 130 comprising a volume modulation mechanism 700 that includes (1) a syringe-style piston 740 having a rigid cylinder 742 and a lockable plunger 744 adapted to create a decreased internal pressure within the volume modulation mechanism 700, and (2) a trigger valve 746, whereby the decreased internal pressure may be transferred to a bladder 200 upon triggering the trigger valve 746 and thereby evacuate fluid 400 from the bladder 200. In such embodiments, the system 130 may be pressurized and depressurized in a way opposite to a pump-action air gun: pumping creates lower pressure (i.e., a vacuum) that is stored in a chamber (e.g., pump 320, or reservoir 600), and the lower pressure is transferred rapidly to the bladder 200 upon activating a trigger 746, such that the lower pressure in the chamber rapidly pulls fluid 400 out of the bladder 200. The trigger 746 may comprise, for instance, a transfer valve 746 comprising a spring valve or a disc valve.

For example, a user may pump to evacuate air 410 from a reservoir 600 and create a lower pressure (i.e., a vacuum) in the reservoir 600 prior to donning the stabilization device 1000, and after donning the device 1000, may trigger a transfer valve 746 that opens the bladder 200 to the vacuum in the reservoir 600, such that the vacuum in the reservoir 600 pulls fluid 400 out of the bladder 200 and into the reservoir 600, thereby triggering a rapid contraction of the bladder 200 around the part of the body wearing the stabilization device 1000. The contraction may happen in a second or less, which a user may perceive as occurring rapidly, compared with the time needed to evacuate other embodiments of a stabilization device 1000, in which the support device 1000 is first donned, and then the bladder 200 is evacuated by a series of individual pumping actions, with the pumping actions occurring after the device 1000 is donned.

As depicted in FIG. 7, in an exemplary embodiment, a rapid-transfer system 130 may comprise a volume modulation mechanism 700 comprising a syringe-style piston 740 having a rigid cylinder 742, a lockable plunger 744, and a trigger valve 746. The rigid cylinder 742 and the lockable plunger 744 function as a reservoir 600, and the trigger valve 746 functions as a reservoir inflow valve 610 as well as a reservoir outflow valve 620. By closing the trigger valve 746, pulling the plunger 744, and locking the plunger 744 in place with an increased volume 714, a vacuum is created in, contained in, and maintained in the rigid cylinder 742 of the syringe-style piston 740. The volume modulation mechanism 700 may be removable from the support device 1000 and detachable from the bladder 200 and fluid coupling 500 for convenient creation of the vacuum prior to replacement on the support device 1000 and re-attachment of volume modulation mechanism 700 to the pump-to-bladder coupling 510. Integration and placement of the volume modulation mechanism 700 on the support device 1000 facilitates convenient access to adjust the integrated adjustable stabilization system 100 on-the-go and on-demand, without the wearer needing to separately store or carry a non-integrated evacuation pump, as in the prior art (see, e.g., non-integrated, detachable “vacuum pump 64” of U.S. Patent Publication 2006/0229541 A1 titled “Orthopedic inlay”).

With the vacuum created, the syringe-style piston 740 may be replaced on the support device 1000 and reattached to the bladder 200 via bladder coupling 510, and the support device 1000 then may be donned by the wearer. Alternatively, after creation of the vacuum, the wearer may first don the support device 1000, and then volume modulation mechanism 700 may be replaced on the support device 1000 and reattached to the bladder coupling 510 and bladder 200. Once the wearer is wearing the support device 1000, the wearer or an assistant may open the bladder valve 310 to open the coupling 510 to the bladder 200, and activate the trigger valve 746 to rapidly evacuate the bladder 200 by pulling the fluid 400 in the bladder 200 into the reservoir 600 of the rigid cylinder 742 of the syringe-style piston 740. The bladder valve 310 may then be reclosed to secure the reduced bladder internal pressure.

The trigger valve 746 may be the bladder valve 310, or they may be separate. If the integrated syringe-style piston 740 is detachable from the support device 1000, an exemplary embodiment may include a trigger valve 746 separate from the bladder valve 310, so that the bladder valve 310 may retain the vacuum in the bladder 200 while the syringe-style piston 740 is prepared and pressurized, such as in preparation to adjust the support device 1000. If the integrated syringe-style piston 740 is not detachable, the trigger valve 746 may be the bladder valve 310, which would first be opened to transfer the vacuum to the bladder 200, and then be closed to securely maintain the vacuum in the bladder 200.

A rapid-transfer system 130 may be helpful when the wearer needs the help of a person other than the wearer, such as a doctor, nurse or therapist, to don the support device 1000. For example, the wearer may be a child; a sleeping, sedated, or comatose patient; a disabled person; an elderly or weak patient; or an immobilized patient in traction. The nurse could prepare the support device 1000 by creating the vacuum prior to placing the support device 1000 on the wearer, then place the support device 1000 on the wearer, and finally trigger the rapid evacuation of the bladder 200 to complete the donning process.

As in the embodiments depicted in FIGS. 6A-6C, the volume modulation mechanism may be conceptualized as having three pump/reservoir positions, such as wherein the reservoir is integrated into the pump and the pump comprises a piston pump or a screw-action pump, wherein the three positions comprise: a neutral pressure position comprising a middle position of a piston or a screw of the pump with a piston chamber or screw chamber of the pump, defining an initial volume 712; a lower pressure position comprising a distal position of the piston within the piston chamber or the screw within the screw chamber, wherein moving the piston or the screw to the distal position increases the reservoir volume within the piston chamber or screw chamber, defining an increased volume 714, and thereby decreases the internal pressure of the reservoir; and a higher pressure position comprising a proximate position of the piston within the piston chamber or the screw within the screw chamber, wherein moving the piston or the screw to the proximate position decreases the reservoir volume within the piston chamber or screw chamber, defining a decreased volume 716, and thereby increases the internal pressure of the reservoir.

FIG. 8 shows a block diagram of another exemplary embodiment of an orthopedic support device 1000 having a thermally-regulatable system 140 comprising an integrated pressure modulation mechanism 300 that is coupled to a bladder 200 and that comprises a reservoir 600, a thermal control device 800, and a circulation device 900, which are adapted to control circulation of a thermally-regulated fluid 400 between the bladder 200 and the integrated pressure modulation mechanism 300. In such embodiments of the present invention, the system 140 may be thermally regulatable for reasons such as providing comfort or therapeutic benefits to the wearer of the device 1000. A thermally-regulatable system 140 may be openable 110 or non-openable 120, and its fluid 400 may comprise a gas 410, a liquid 420, or a gas-liquid mixture.

A thermally-regulatable system 140 may include one or more of a thermal control device 800 (e.g., heating device, a cooling device, a heating/cooling device, or a heat exchanger), a circulation device 900, and an optional pressure-cycling device 920. A thermal control device 800 may be adapted to heat and/or cool the fluid 400 before/during/after pressurization of the bladder 200, such as to warm the bladder 200 and the beads 210 prior to evacuation, or fill the bladder 200 with warmed or cooled fluid 400 when not evacuated. The thermal control device 800 may be adapted to automate thermal control/regulation of the fluid 400 through the system 140. The thermal control device 800 may comprise, for instance, electronic circuitry and a programmable microprocessor to control the thermal control/regulation functionality. Likewise, the circulation device may comprise, for example, a power supply (e.g., battery, adapter, and/or converter) and a heating element and/or a cooling element, powered by the power supply, and controlled by the microprocessor.

When being worn by a wearer, the bladder 200, beads 210, and fluid 400 may warm due to body heat of a wearer, and a heat exchanger may be adapted to equalize a fluid temperature with an ambient temperature. A heat exchanger need not be powered and may provide a lower-power, lower-cost alternative to a powered heating or cooling device. An exemplary heat exchanger might comprise couplings 500 or a reservoir 600 on the outer side of the support device 1000, distal from the heat generated by the wearer's body.

A circulation device 900 may be adapted to circulate fluid 400 between the bladder 200 and the reservoir 600 when the bladder 200 is not pressurized, such as to circulate the fluid 400 through a thermal control device 800 (e.g., a heating/cooling device or a heat exchanger). The circulation device 900 is adapted to automate circulation of the fluid 400 through the system 140. The circulation device 900 may be adapted to circulate the fluid 400 through the thermal control device 800 without pressurizing the bladder 200 during circulation. The circulation device 900 may comprise a part of an integrated pressure modulation mechanism 300 and may be adapted to control and power circulation of fluid 400 within the system 140 by controlling and powering the evacuation pump 320 and the valves 310, 324, 328, 610, 620. Likewise, the circulation device 900 may be adapted to effectuate the pressurization of the bladder 200 by evacuating the fluid 400 from the bladder 200.

The circulation device 900 may comprise, for instance, electronic circuitry and a programmable microprocessor to control the circulation functionality. Likewise, the circulation device may comprise, for example, a power supply (e.g., battery, adapter, and/or converter) and electric couplings 910 to an electrically-powered thermal control device 800, an electrically-powered evacuation pump 320, and electrically-powered valves 310, 324, 328, 610, 620. The pump 320 and valves 310, 324, 328, 610, 620 may include, for example, electrically-powered solenoids to actuate the evacuation pump 320 and the valves 310, 324, 328, 610, 620. Moreover, the thermal control device 800, the circulation device 900, and optional pressure-cycling device 920 may be adapted to interface via wired or wireless communication with a detached computing device (e.g., laptop, tablet, smartphone, smartwatch, etc.) for programming, diagnostics, and data uploads or downloads (i.e., via an app or other software). The thermally-regulatable system 140 may comprise a pressure-cycling system 150, as well, or a pressure-cycling system 150 may be implemented without a thermal control device 800.

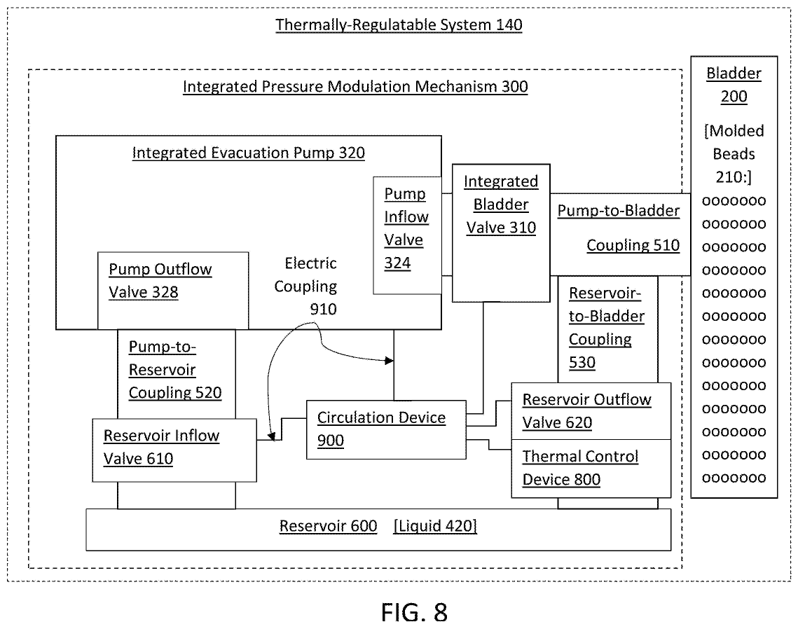

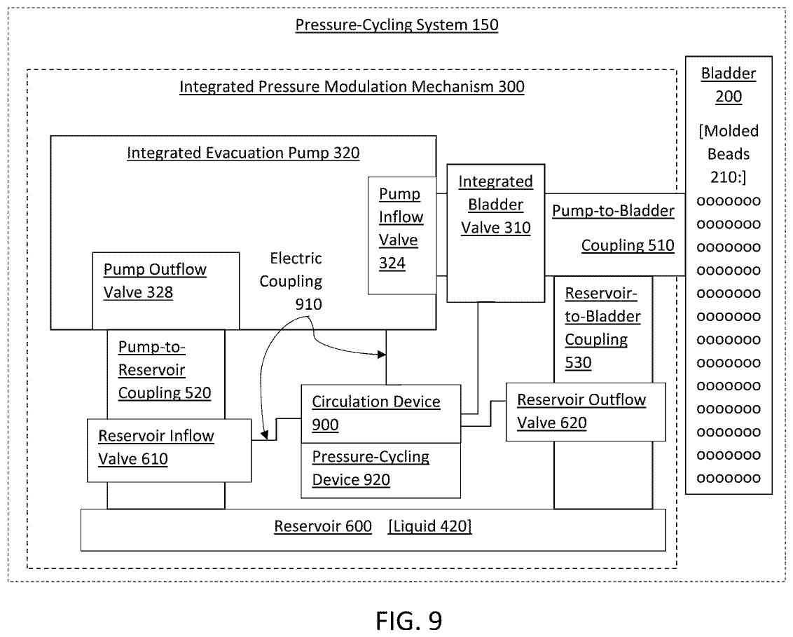

FIG. 9 shows a block diagram of another exemplary embodiment of an orthopedic support device 1000 having a pressure-cycling system 150 comprising an integrated pressure modulation mechanism 300 that is coupled to a bladder 200 and that comprises a reservoir 600, a circulation device 900, and a pressure-cycling device 920, which are adapted to cycle pressure of, and control circulation of, a liquid 420 between the bladder 200 and the integrated pressure modulation mechanism 300. A pressure-cycling system 150 may comprise a pressure-cycling device 920 that may be adapted to automatically cycle the bladder internal pressure between being pressurized and not pressurized to allow for associated actions, including circulating heated or cooled liquid, periodic relief of pressure applied by the rigidity of the molded beads 210 in an evacuated bladder 200, and/or rhythmically massaging the part of the body wearing the stabilization device 1000 through the bladder 200 alternating between increasing and decreasing levels of rigidity from rigid arrangements 210″. The pressure-cycling device 910 is adapted to automate cycling of pressure within the system 150, and wherein cycling of pressure within the system 150 comprises repeating a pressure cycle of evacuating the bladder 200, maintaining the bladder 200 at the decreased bladder internal pressure, and increasing the bladder internal pressure from the decreased bladder internal pressure.

The pressure-cycling device 920 may comprise a circulation device 900 with additional cycling functionality to complement circulation functionality, such as through use of correspondingly-programmed hardware or software (i.e., via a display on the boot, or an app, a user interface, or other software on a computing device). For instance, in some situations, circulation of fluid 400 may be performed, with or without thermal regulation, without pressuring the bladder 200, thereby allowing a loose arrangement 210′ of beads 210; while in other situations, circulation of fluid 400 may be performed, with or without thermal regulation, by pressurizing the bladder 200 to form a rigid arrangement 210″ of beads 210. In situations having pressure-cycling and thermal regulation, for example, the fluid 400 outside the bladder 200 may be heated or cooled while the bladder 200 is maintained in an evacuated state having a decreased bladder internal pressure and rigid arrangement 210″ of beads 210. When the fluid 400 outside the bladder 200 is sufficiently heated or cooled, the bladder 200 may be depressurized, and the fluid 400 may be circulated through the bladder 200 for comfort or therapeutic effect. After a threshold is reached (e.g., time, fluid temperature, pressure, etc.), the bladder 200 may be evacuated and the fluid 400 drawn back to the reservoir 600.

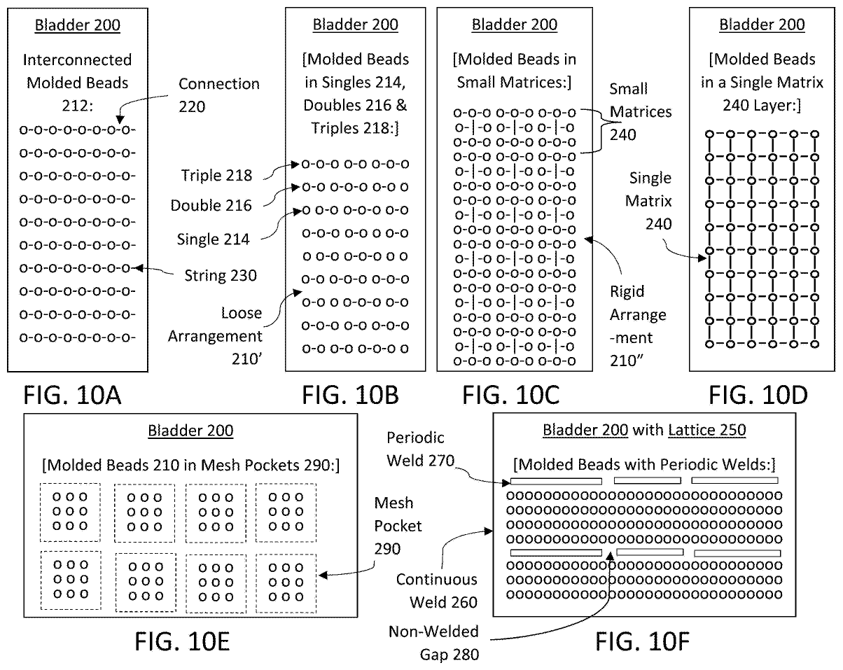

FIGS. 10A-10F show block diagrams of exemplary combinations of bladders 200 and molded beads 210. Some molded beads may include connections 220 between beads 210 to form groups of beads, such as interconnected beads 212. Groups of beads may include one or more of various combinations of beads 210, such as interconnected beads 212, bead singles 214, bead doubles 216, bead triples 218, strings 230 of multiple beads 210, and matrices 240 of multiple beads 210. When a bladder 200 is not pressurized under a partial vacuum, the beads 210 typically will form loose arrangements 210′ of beads 210. When a bladder 200 is pressurized under a partial vacuum, the beads 210 are adapted to form rigid arrangements 210″ of beads 210. The configuration of the bladder 200 also may impact the arrangement of the beads 210. For instance, a bladder 200 may have an internal lattice 250, in which the bladder 200 has a perimeter seal defined by one or more continuous welds 260, and intermittent interior seals formed by periodic welds 270 separated by non-welded gaps 280. In some embodiments, a bladder 200 may include beads 210 within mesh pockets 290 that allow the fluid 400 to pass through the mesh pockets 290 to enable evacuation of the bladder 200.

In some embodiments, the beads 210 may be loose and free to move within the bladder 200 when the bladder 200 is not evacuated. Loose beads 210 that are free to move within an unevacuated bladder 200 may be easily moved within the unevacuated bladder 200, for better and for worse. In other embodiments, the molded beads may be grouped with the bladder to reduce risks of undesired clumping of the beads 210, or to predispose the molded beads to consolidate into a particular shape when the bladder 200 is evacuated. Depending on the intended use and wearing location of the orthopedic support device 1000, the number and type of bead configurations may vary, and those configurations may include combinations having one or more types, including interconnected beads 212, single 214 beads 210, pairs 216 of beads 210, strings 230 of multiple beads, and a matrix 240 or matrices 240 of interconnected beads 212. In addition, the beads themselves may be of various configurations, shapes, sizes, materials, properties, and fabrication.

As referenced herein, the term molded bead encompasses any suitable bead, ball, microbead, or other small three-dimensional object smaller than 5 millimeters across its largest dimension. Beads 210 may have various properties, chosen for the particular orthopedic support device 1000, such as hard, soft, dense, non-dense, squishy, compressible, non-compressible, slippery, edgy, spherical, non-spherical, stackable, non-stackable, solid, hollow, porous, non-porous, regular, irregular, etc. For example, small cubic beads would be stackable, whereas small spherical beads would be non-stackable, and stackable beads might form a denser rigid arrangement 210″ than non-stackable beads would. Likewise, the selection of materials and fabrication techniques for fabrication of the beads 210 depends on the particular orthopedic support device 1000 and the desired properties of the beads 210 chosen for the device 1000. For example, beads 210 may be formed of thermoplastics, elastomers, ceramics, foams, metals, woods, gels, crystals, amorphous materials, glass, etc., and the material selection may be impacted by the type of fluid 400 chosen, such as air 410, or a liquid 420 (e.g., water, oil, hydraulic fluid, etc.), and how much weight or wear will be applied to the beads during use.

Grouping of the molding beads 210 may take one or more forms, including interconnecting the beads 210 with connections 220 comprising, for example, string, fibers, filaments, or molded interconnections that are introduced when the molded beads 210 are formed initially. For example, a string or fiber may be placed between molds used to form the beads 210, and the molds then may be filled with a thermoplastic or elastomer to form the beads 210, which consequently have the string or fiber embedded in them and interconnecting them, forming strings 230 not unlike strings of beads used in necklaces for Mardi Gras or party favors. Instead of string, the molds themselves may form thin channels between bead molds that interconnect the beads 210 once the molds, including the channels, are filled with thermoplastic or elastomer and cured.

In other embodiments, the beads 210 may be finely interconnected with filaments to form a quasi-matrix 240 of beads 210, wherein the quasi-matrix 240 of beads 210 collapses into a consistent shape when the bladder 200 is evacuated and a partial vacuum is created within the bladder 200. The filament connections 220 may exhibit elasticity, plasticity, both, or neither, wherein the filaments form a quasi-matrix 240 of beads 210 that may be analogous a spider's web full of gnats. Formation of a quasi-matrix 240 of beads 210 might occur by laying a single layer of beads 210 onto a sheet having regularly-spaced bead-sized indentations into which the beads 210 rest, and drizzling horizontal and vertical lines of dissolved elastomer (e.g., spandex or Lycra®) over the beads 210. Once the elastomer cures, a single-bead-thick layer matrix 240 is formed, and several of these layer matrices 240 may be layered on top of each other to form a quasi-matrix 240 having several layers of beads 210, comprising a stack of beads 210 several beads 210 thick and dozens, hundreds, or thousands of beads 210 across vertically and horizontally. Stacking layers of bead matrices of differing shapes may create a desired three-dimensional shape in a fashion similar to using stereolithography, i.e., three-dimensional (3-D) printing, which may be used also.

In some embodiments, the configuration of the bladder 200 may predispose the molded beads 210 to form groups or consolidate into a particular shape when the bladder 200 is evacuated. For instance, beads 210 may be enclosed within a lattice 250 formed by periodic welding of a top layer and a bottom layer of the bladder 200. The lattice 250 may take various configurations, such as layers, channels, honeycombs, etc. In contrast to periodic welding, a continuous weld 260 typically forms the outer perimeter seams of the bladder 200. Periodic welding forms a periodic weld 270 of the top layer and the bottom layer of the bladder 200. Periodic welds 270 also can be referred to as periodic welds, mid-point welds, discontinuous welds, and intermittent welds. A lattice 250 defined by periodic welds 270 may limit the mobility of the beads 210 within the bladder 200, while allowing fluid 400 to pass through the lattice 250 via non-welded gaps 280 to allow the bladder to be pressurized (i.e., evacuated) and de-pressurized. Non-welded gaps 280 may be small enough to prevent beads from leaving an area, or the non-welded gaps 280 may be large enough to allow beads 210 to leave an area in a restricted or unrestricted fashion.

In another example, beads 210 may be enclosed within mesh pockets 290 within the bladder 200, wherein the mesh pockets 290 limit the mobility of the beads 210 within the bladder 200. The mesh pockets 290 allow fluid 400 to pass through the mesh pocket 290 to allow the bladder 200 to be evacuated and depressurized. Mesh pockets 290 may be layered and subdivided as well, to define desired shapes and three-dimensional configurations of interconnected beads 212, loose single beads 214, or a combination thereof. The mesh pockets 290 may be loose within the bladder 200, fixed in place within the bladder 200, or a combination thereof.

Numerous possible embodiments of the orthopedic support device 1000 are envisioned, such as orthopedic walkers, arm braces, wrist braces, ankle braces, leg braces, knee braces, hand braces, elbow braces, shoulder braces, neck braces, and back braces. Other embodiments may include orthotic shoes and boots, such as running shoes, hiking boots, or ski boots. An advantage of the integrated adjustable stabilization system 100 in the orthopedic support device 1000 is that that system 100 may be adjusted repeatedly to conform to a given wearer's body part, and the same device 1000 may then be readjusted repeatedly to conform to a different wearer's body part. Alternating between a loose arrangement 210′ of beads 210 and a rigid arrangement 210″ of beads 210 requires minimal effort, and allows for the potential for a good fit with each wear and wearer, even as a wearer's own body part may swell or contract, such as due to exercise, weight gain/loss, medications, etc. Integrating the integrated pressure modulation mechanism directly into the orthopedic support device 1000 allows the device 1000 to be adjusted, donned, removed, and re-donned, etc., without a wearer needing to remember to carry and store an external, non-integrated evacuation pump, as is the case in the prior art, such as “vacuum pump 64” of U.S. Patent Publication 2006/0229541 A1 titled “Orthopedic inlay.”

### Exemplary Preferred Embodiments of the Invention

In the FIGS. 11-17 discussed below, an exemplary preferred embodiment of the orthopedic support device 1000 is presented that comprises an orthopedic walker 1100.

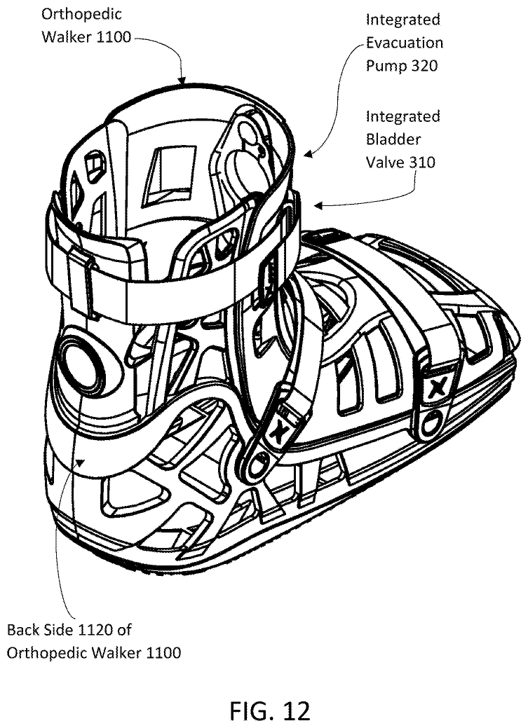

FIG. 11 shows a front side perspective view of an exemplary embodiment of the orthopedic support device 1000 comprising an orthopedic walker 1100 having an integrated adjustable stabilization system 100 comprising an openable and closable system 110 having an integrated pressure modulation mechanism 300 that comprises an integrated bladder valve 310 and an integrated evacuation pump 320. The integrated bladder valve 310 and the integrated evacuation pump 320 are positioned on a front side 1110 of the orthopedic walker 1100. The orthopedic walker 1100 shown is a hard-shell assembly 1130 of interconnecting parts 1140 made of durable, rigid plastic. The orthopedic walker 1100 includes adjustable straps 1150 at or near a wearer's toes, instep, and calf and/or shin to allow for a secure fit when worn. FIGS. 12-14 respectively show a rear side perspective view, a front elevation view, and a side elevation view of the orthopedic walker shown in FIG. 11.

Not shown is an interior lining that fits inside the hard-shell assembly 1130 and is removable from the walker 1100. Interior linings as such are well-known in the prior art and not the focus of the present invention. The interior lining preferably includes one or more bladders 200 containing beads 210 and a pump-to-bladder coupling 510, which may be attached to and detached from the integrated bladder valve 310. The configuration of the hard-shell assembly 1130 determines where one or more bladders 200 containing beads 210 will fit within the orthopedic walker 1100. Various configurations of bladders (without beads) and bladder couplings are known in the prior art, such as shown in FIGS. 27-32 of U.S. Pat. No. 8,02,724 B2 titled “Circumferential Walker” for use in an orthopedic walker. Various configurations of bladders with molded beads for use in an orthopedic walker are disclosed in U.S. Patent Publication 2006/0229541 A1 titled “Orthopedic inlay.” A comparable bladder 200 similarly fitted to the hard-shell assembly 1130 may be coupled to the integrated bladder valve 310 of FIG. 11 using a comparable pump-to-bladder coupling 510.

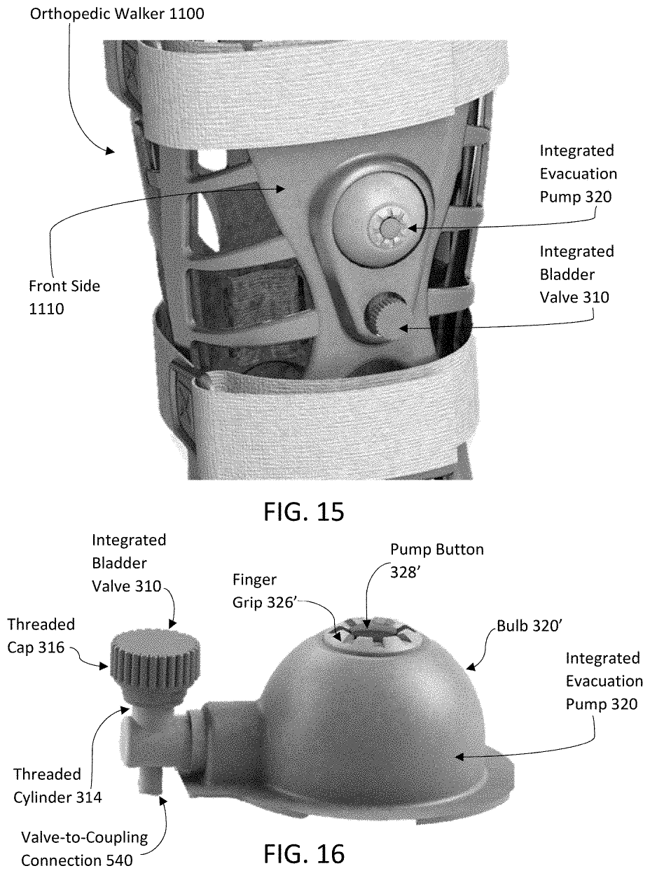

FIG. 15 shows a front perspective partial view of another exemplary embodiment of an orthopedic support device 1000 comprising an orthopedic walker 1100 having an integrated adjustable stabilization system 100 comprising an openable and closable system 110 having an integrated pressure modulation mechanism 300 that comprises an integrated bladder valve 310 and an integrated evacuation pump 320.

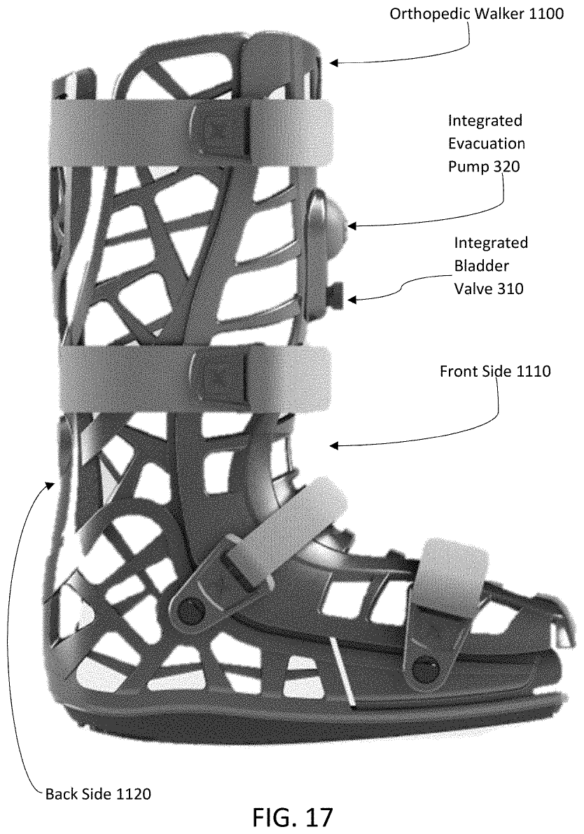

FIG. 16 shows a side perspective view of the exemplary integrated bladder valve 310 and the exemplary integrated evacuation pump 320 that are integrated into the orthopedic walker 1100 depicted in FIG. 15. Similarly, FIG. 17 shows a side elevation view of the orthopedic walker 1100 depicted in FIG. 15.

As shown in FIG. 16, the integrated bladder valve 310 includes a threaded cylinder 314 and a threaded cap 316 that may be turned to allow air 410 to enter a pump-to-bladder coupling 510 (not shown in FIG. 16) through a valve-to-coupling connection 540 that is adapted to be connected to the pump-to-bladder coupling 510. As shown, the integrated evacuation pump 320 includes a bulb 320′, a grooved/notched finger grip 326′, and a pump button 328′. The bulb 320′ serves as a chamber of the integrated evacuation pump 320. The finger grip 326′ serves as a pump output port 326, and the grooves or notches vent air when a finger covers the finger grip 326′. The pump button 328′ serves as a pump outflow valve 328. The finger grip 326′ on the bulb 320′ allows air 410 to escape the bulb 320′ when the bulb 320′ and pump button 328′ are depressed. Pumping action includes forcing air 410 out of the evacuation pump 320 by depressing both the pump button 328′ and the bulb 320′ to deform the bulb 320′ from a bulb shape to a deformed shape. When the pump button 328′ and bulb 320′ are released, the pump button 328′ closes the pump outflow valve 328, and the deformed bulb 320′ returns to the bulb shape and draws air 410 through an opened bladder valve 310, through the valve-to-coupling connection 540, and, if the valve-to-coupling connection 540 were connected to a pump-to-bladder coupling 510, out of a bladder 200 coupled to bladder valve 310.

Prior to donning an orthopedic walker 1100 of FIGS. 11-14 and 15-17, a wearer would insert an interior lining into the walker 1100, wherein the interior lining includes at least one bladder 200, beads 210 in the bladder 200, and a pump-to-bladder coupling 510, and attach the pump-to-bladder coupling 510 to the valve-to-coupling connection 540 at an interior side of the walker 1100, partially visible in FIG. 12. With the interior lining in place, the bladder 200 and beads 210 properly positioned, and the pump-to-bladder coupling 510 connected to the valve-to-coupling connection 540, the wearer may don the walker 1100, open the integrated bladder valve 310, use the integrated evacuation pump 320 to evacuate the bladder 200 and form a rigid arrangement 210″ of beads 210, and close the integrated bladder valve 310 to maintain the rigid arrangement 210″ in the evacuated bladder 200. To remove the walker 1100, the wearer may open the integrated bladder valve 310 to allow air 410 to reinflate and depressurize the bladder 200, loosen the adjustable straps 1150, and take off the walker 1100.

The foregoing description discloses exemplary embodiments of the invention. While the invention herein disclosed has been described by means of specific embodiments and applications thereof, numerous modifications and variations could be made thereto by those skilled in the art without departing from the scope of the invention set forth in the claims. Modifications of the above disclosed apparatus and methods that fall within the scope of the claimed invention will be readily apparent to those of ordinary skill in the art. Accordingly, other embodiments may fall within the spirit and scope of the claimed invention, as defined by the claims that follow hereafter.

In the description above, numerous specific details are set forth in order to provide a more thorough understanding of embodiments of the invention. It will be apparent, however, to an artisan of ordinary skill that the invention may be practiced without incorporating all aspects of the specific details described herein. Not all possible embodiments of the invention are set forth verbatim herein. A multitude of combinations of aspects of the invention may be formed to create varying embodiments that fall within the scope of the claims hereafter. In addition, specific details well known to those of ordinary skill in the art have not been described in detail so as not to obscure the invention. Readers should note that although examples of the invention are set forth herein, the claims, and the full scope of any equivalents, are what define the metes and bounds of the invention protection.

What is claimed is:

1. An orthopedic support device adapted for stabilizing a part of a body of a wearer, the orthopedic support device comprising an integrated adjustable stabilization system, wherein the stabilization system comprises an integrated pressure modulation mechanism adapted to be coupled with and evacuate a bladder containing a plurality of molded beads, wherein the integrated pressure modulation mechanism is adapted to be coupled with the bladder via a bladder coupling, and wherein the integrated pressure modulation mechanism is adapted to evacuate a fluid from the bladder through the bladder coupling to decrease a bladder internal pressure to a decreased bladder internal pressure, consolidate the plurality of molded beads from a loose arrangement to a rigid arrangement, and maintain the bladder internal pressure at the decreased bladder internal pressure.

2. The orthopedic support device of claim 1, wherein the orthopedic support device comprises an orthopedic walker, an arm brace, a wrist brace, an ankle brace, a leg brace, a knee brace, a hand brace, an elbow brace, a shoulder brace, a neck brace, or back brace.

3. The orthopedic support device of claim 1, wherein the integrated adjustable stabilization system comprises an openable and closable system, and wherein the fluid comprises a gas.

4. The orthopedic support device of claim 1, wherein the integrated adjustable stabilization system comprises a non-openable closed system, and wherein the fluid comprises a liquid.

5. The orthopedic support device of claim 1, wherein the integrated pressure modulation mechanism further comprises an integrated evacuation pump and an integrated bladder valve, wherein the integrated evacuation pump is coupled to the integrated bladder valve, and the integrated bladder valve is adapted to be coupled to the bladder coupling, and wherein the bladder coupling comprises a pump-to-bladder coupling that is adapted to be coupled to the bladder.

6. The orthopedic support device of claim 1, wherein the integrated pressure modulation mechanism comprises an integrated evacuation pump, an integrated bladder valve, a reservoir, a pump-to-reservoir coupling that couples the integrated evacuation pump and the reservoir, and a reservoir-to-bladder coupling that is adapted to couple the reservoir and the bladder coupling; wherein after coupling the integrated pressure modulation mechanism with the bladder via the bladder coupling, and after opening the integrated bladder valve, the integrated evacuation pump is adapted to evacuate the fluid from bladder through the bladder coupling and into the reservoir through the pump-to-reservoir coupling; and wherein the integrated pressure modulation mechanism is adapted to allow the fluid to return from the reservoir through the reservoir-to-bladder coupling and through the bladder coupling to the bladder to increase the bladder internal pressure from the decreased bladder internal pressure.

7. The orthopedic support device of claim 1, wherein the integrated pressure modulation mechanism further comprises a volume modulation mechanism and an integrated bladder valve, wherein the volume modulation mechanism is coupled to the integrated bladder valve, and the integrated bladder valve is adapted to be coupled to the bladder coupling, and wherein the bladder coupling comprises a pump-to-bladder coupling that is adapted to be coupled to the bladder.

8. The orthopedic support device of claim 1, wherein the integrated adjustable stabilization system comprises a rapid-transfer system, wherein the integrated pressure modulation mechanism further comprises a reservoir and a trigger valve, wherein the integrated pressure modulation mechanism is adapted to evacuate the reservoir and to maintain the reservoir at a decreased reservoir internal pressure, and wherein the trigger valve is adapted to be coupled to the bladder coupling and, after coupling the trigger valve to the bladder coupling, to rapidly evacuate the fluid from bladder through the bladder coupling upon opening the trigger valve.

9. The orthopedic support device of claim 1, wherein the integrated adjustable stabilization system comprises a thermally-regulatable system, wherein the thermally-regulatable system further comprises a thermal control device and a circulation device, wherein the thermal control device is adapted to regulate a temperature of the fluid in the system, and wherein the circulation device is adapted to automate circulation of the fluid through the system.

10. The orthopedic support device of claim 1, wherein the integrated adjustable stabilization system comprises a pressure-cycling system, wherein the pressure-cycling system further comprises a circulation device and a pressure-cycling device, wherein the circulation device is adapted to automate circulation of the fluid through the system, wherein the pressure-cycling device is adapted to automate cycling of pressure within the system, and wherein cycling of pressure within the system comprises repeating a pressure cycle of evacuating the bladder, maintaining the bladder at the decreased bladder internal pressure, and increasing the bladder internal pressure from the decreased bladder internal pressure.

11. The orthopedic support device of claim 1, the integrated adjustable stabilization system further comprising the bladder, the plurality of molded beads, and the bladder coupling.

12. The orthopedic support device of claim 11, wherein the plurality of molded beads comprises at least one group of beads and comprises connections between the beads in a group.

13. The orthopedic support device of claim 11, wherein the bladder further comprises at least one mesh pocket containing molded beads.

14. The orthopedic support device of claim 11, wherein the bladder further comprises a lattice formed by at least one periodic weld in the bladder having at least one non-welded gap defined by the periodic weld.

15. A method for stabilizing a part of a body of a wearer, the method comprising:
preparing an orthopedic support device for donning on the part of the body of the wearer, wherein the orthopedic support device comprises an integrated adjustable stabilization system; wherein the stabilization system comprises an integrated pressure modulation mechanism, a bladder, a plurality of molded beads inside the bladder, and a bladder coupling; wherein the bladder coupling couples the bladder with the integrated pressure modulation mechanism; and wherein the integrated pressure modulation mechanism is adapted to evacuate a fluid from the bladder to decrease a bladder internal pressure to a decreased bladder internal pressure, consolidate the plurality of molded beads from a loose arrangement to a rigid arrangement, and maintain the bladder internal pressure at the decreased bladder internal pressure;
donning the orthopedic support device;
evacuating the bladder using the integrated pressure modulation mechanism to consolidate the plurality of molded beads from the loose arrangement to the rigid arrangement; and
maintaining the bladder in an evacuated state having the decreased bladder internal pressure.

16. The method of claim 15, wherein the integrated adjustable stabilization system further comprises a rapid-transfer system, wherein the integrated pressure modulation mechanism further comprises a reservoir and a trigger valve, wherein the integrated pressure modulation mechanism is adapted to evacuate the reservoir and to maintain the reservoir at a decreased reservoir internal pressure, and wherein the trigger valve is adapted to be coupled to the bladder coupling and, after coupling the trigger valve to the bladder coupling, to rapidly evacuate the fluid from bladder through the bladder coupling and into the reservoir upon opening the trigger valve, wherein the method further comprises:
evacuating the reservoir before evacuating the bladder;
maintaining the reservoir at the decreased reservoir internal pressure after evacuating the reservoir and before evacuating the bladder;
coupling the trigger valve to the bladder coupling before evacuating the bladder; and,
opening the trigger valve to rapidly evacuate the fluid from bladder through the bladder coupling and into the reservoir upon opening the trigger valve.

17. The method of claim 16, wherein donning the orthopedic support device occurs:
(a) after evacuating the reservoir, maintaining the reservoir at the decreased reservoir internal pressure, and coupling the trigger valve to the bladder coupling; and
(b) before opening the trigger valve to rapidly evacuate the fluid from the bladder.

18. The method of claim 15, wherein the integrated adjustable stabilization system comprises a thermally-regulatable system, wherein the thermally-regulatable system further comprises a thermal control device and a circulation device, wherein the thermal control device is adapted to regulate a temperature of the fluid in the system, and wherein the circulation device is adapted to automate circulation of the fluid through the system; wherein the method further comprises:
regulating the temperature of the fluid in the system; and
automatically circulating the fluid through the system.

19. The method of claim 15, wherein the integrated adjustable stabilization system comprises a pressure-cycling system, wherein the pressure-cycling system further comprises a circulation device and a pressure-cycling device, wherein the circulation device is adapted to automate circulation of the fluid through the system, wherein the pressure-cycling device is adapted to automate cycling of pressure within the system, and wherein cycling of pressure within the system comprises repeating a pressure cycle of evacuating the bladder, maintaining the bladder at the decreased bladder internal pressure, and increasing the bladder internal pressure from the decreased bladder internal pressure; wherein the method further comprises:
automatically circulating the fluid through the system; and
automatically cycling the pressure of the fluid in the system.

20. An orthopedic support device adapted for stabilizing a part of a body of a wearer, the orthopedic support device comprising an orthopedic walker comprising an integrated adjustable stabilization system; wherein the stabilization system comprises:
a bladder;
a plurality of molded beads contained in the bladder;
a bladder coupling coupled to the bladder; and,
an integrated pressure modulation mechanism adapted to be coupled with and evacuate the bladder containing the plurality of molded beads, wherein the integrated pressure modulation mechanism is adapted to be coupled with the bladder via the bladder coupling, and wherein the integrated pressure modulation mechanism is adapted to evacuate a fluid from the bladder through the bladder coupling to decrease a bladder internal pressure to a decreased bladder internal pressure, consolidate the plurality of molded beads from a loose arrangement to a rigid arrangement, and maintain the bladder internal pressure at the decreased bladder internal pressure;
wherein the integrated pressure modulation mechanism comprises an integrated evacuation pump and an integrated bladder valve; wherein the integrated evacuation pump is coupled to the integrated bladder valve, and the integrated bladder valve is adapted to be coupled to the bladder coupling; and wherein the bladder coupling comprises a pump-to-bladder coupling that is adapted to be coupled to the bladder.
